(19) United States
(12) Patent Application Publication
(10) Pub. No.: US 2024/0042279 A1
(43) Pub. Date: Feb. 8, 2024

(54) MULTI-VENUE VIRTUAL REALITY MOVEMENT TRACKING SYSTEM

(71) Applicant: Barry French, JR., Bay Village, OH (US)

(72) Inventor: Barry French, JR., Bay Village, OH (US)

(21) Appl. No.: 18/113,915

(22) Filed: Feb. 24, 2023

Related U.S. Application Data
(60) Provisional application No. 63/313,297, filed on Feb. 24, 2022.

(51) Int. Cl. A63B 24/00; A63B 71/06

(52) U.S. Cl. CPC A63B 24/0062; A63B 71/0622; A63B 24/0003; A63B 2071/0625; A63B 2071/0638; A63B 2220/05; A63B 2225/20

(57) ABSTRACT

A system that tracks physical activity over multiple venues to assess and track the performance of a user. Physical assessments from the different venues is tracked in a same profile of a user that is stored in, for example, a cloud-based system. The progress of the user is tracked, analyzed, and reported to facilitate a holistic tracking of progress towards personal and clinical goals.

### CROSS REFERENCE TO RELATED APPLICATIONS

This application claims priority to U.S. Provisional Patent Application No. 63/313,297, filed on Feb. 24, 2022, entitled “Multi-Venue Virtual Reality Movement Tracking System,” which is incorporated herein by reference in its entirety.

### TECHNICAL FIELD

The present invention is generally related to physical assessment and tracking movements in a virtual space and, more specifically, assessing physicality across multiple site-based virtual spaces.

### BACKGROUND

As people (e.g., professional, amateur, and/or home athletes, etc.) strive to prove their physical performance and achieve desired health outcomes, they set goals to help develop in various areas. However, these athletes, their clinicians, and their trainers, etc., lack a way to quantify and qualify their current performance and track their improvements over time. This can be especially difficult when the athletes work with multiple clinicians and/or trainers and perform self-training at different venues.

### SUMMARY

Described herein is a system that tracks physical activity over multiple venues to assess and track the performance of a user. The movement tracking system at each venue includes similar movement tracking hardware (e.g., a camera system and a display, etc.) to prompt user motion, and track and quantify responsive user movement. Each venue may use a different user interface (“UI”) (sometimes referred to as a “portal”) based on the purpose of the activity of the venue. For example, a clinician or physical therapist may use a portal that is specific to clinical requirements to treat the user's injuries (e.g., appointment tracking, clinically specific workouts, insurance tracking, billing, other relevant medical data, links to other medical tracking systems, HIPPA compliance, etc.). As another example, a fitness center and/or gym may use a different portal that tracks and quantifies workout data and performance and may provide some non-sensitive information to a personal trainer, but does not include more personal clinical information. As another example, a user may access a home portal that allows the user to customize the interface to include the gym interface and at least some aspects of the clinical interface. When the user profile is associated with a gym (e.g., through a membership system), the home portal may provide access to gym-specific content, such as access to audio and/or visual communication with a trainer associated with the gym. When associated with a clinic, the home portal may provide access to clinically specific content and/or audio/visual communication with the physical therapist. The physical assessment from the different venues is tracked in the same profile of the user that is stored in, for example, a cloud-based system. Non-related devices, such as a phone, tablet, smart watch, or mobile device can be used to access the user's profile data. Aspects of training/exercise unrelated to the movement tracking system, such as a recommended exercise routine or independent workout, may be recorded as part of the user's profile. The progress of the user is tracked, analyzed, and reported to facilitate a holistic tracking of progress towards personal and clinical goals.

### BRIEF DESCRIPTION OF THE DRAWINGS

Operation of the present disclosure may be better understood by reference to the following detailed description taken in connection with the following illustrations, wherein:

FIG. 1 is a conceptual diagram of an example system to provide to a holistic approach to fitness and/or rehabilitation through integrated analytics across multiple venues, in accordance with the teachings of this disclosure.

FIG. 2-21 illustrate example interfaces to provide information contained in the profile of the user depending on the venue at which the profile is being viewed, in accordance with the teachings of this disclosure.

### DETAILED DESCRIPTION

Reference will now be made in detail to exemplary embodiments of the present disclosure, examples of which are illustrated in the accompanying drawings. It is to be understood that other embodiments may be utilized and structural and functional changes may be made without departing from the respective scope of the present disclosure. Moreover, features of the various embodiments may be combined or altered without departing from the scope of the present disclosure. As such, the following description is presented by way of illustration only and should not limit in any way the various alternatives and modifications that may be made to the illustrated embodiments and still be within the spirit and scope of the present disclosure.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

As described below, provided is a performance system that tracks physical activity over multiple venues to assess and track the performance of a user to facilitate the user obtaining their goals. These goals may be, for example, physical or cognitive therapeutic goals, performance or fitness goals, and/or health goals. The performance system may communicatively couple to movement tracking hardware located at different venues. The movement tracking hardware includes a user interface (“UI”) (sometimes referred to as a “portal”) that has access to different parts of the profile depending on the type of the venue. For example, there may be a clinical portal, a gym or fitness portal, and/or a home portal, etc. The user is able to connect their profile to the movement tracking hardware located at a particular venue to facilitate working on their goal at that venue.

The profile includes biographic data (e.g., weight, height, age, etc.), sports and fitness medical history, treatment history, exercise history, and analytical data, etc. The portion of the profile available on a particular movement tracking hardware (via the portal) depends on the role assigned to the movement tracking hardware. For example, movement tracking hardware in a clinical setting may have access to the full profile, while movement tracking hardware in a gym may have access to a portion of the profile. As another example, the movement tracking hardware in a home setting may have access to all of, or to subsets of the clinical portions and the gym portions of the profile.

The system tracks analytics over multiple sessions to provide a quantitative measurement to improvement of performance of the user over time, considering activity performed at different venues, with different programs, and/or different goals for improvement. A portion of these analytics viewable by the user may be different depending on the type of portal being to view the analytics. Additionally, the venue may provide different programs via different portals depending on associations with organizations (e.g., clinics, gyms, etc.) associated with the user profile.

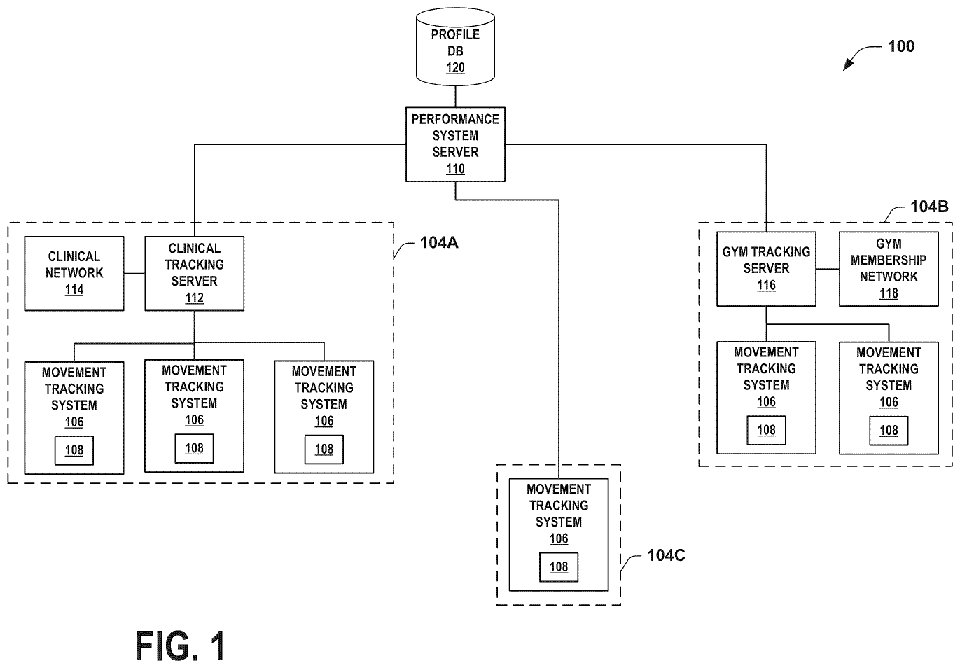

FIG. 1 illustrates an example system 100 to provide a holistic approach to fitness and/or rehabilitation through integrated analytics across multiple venues 104A, 104B, and 104C with one or more movement tracking systems 106. The movement tracking systems 106 at the different venues 104A, 104B, and 104C may include different user interfaces (sometimes referred to as “portals”) 108 that provide different content depending on the use of the movement tracking systems 106 at the particular venue 104A, 104B, and 104C. The system 100 includes a performance system server 110 that provides a unified platform for analytics to quantitatively evaluate performance of a user across different activities at the multiple venues 104A, 104B, and 104C to, for example track performance, identify potential issues, and track physical rehabilitation and/or therapy.

In the illustrated example, the venues include a rehabilitation clinic 104A, a gym and/or fitness center 104B, and a home 104C of the user. In the illustrated example, the clinic 104A includes one or more of the movement tracking systems 106 with which a user performs exercises as part of physical rehabilitation. In the illustrated example, the rehabilitation clinic 104A includes a clinical tracking server 112. Alternatively, the clinical tracking server 112 may be hosted at or integrated into the performance system server 110. The clinical tracking server 112 provides the portal 108 for the movement tracking systems 106, exchanges data with the performance system server 110, and acts to integrate the movement tracking systems 106 into a clinical network 114 that contains clinic specific data used by the portal 108, such as billing information, medical history, and/or insurance, etc. As described below, a user registers with the operator of the clinic 104A to connect their profile managed by the performance system server 110. Through the portal 108, the user and clinician select programs to workout targeted areas of the body, review progress as tracked by the performance system server 110 and interact with other clinic specific information. The performance system server 110 collects this workout data to analyze.

In the illustrated example, the venues include a gym 104B. For example, the gym 104B may be a commercially operated gym with multiple locations, each with one or more of the movement tracking systems 106. In the illustrated example, the gym 104B includes one or more of the movement tracking systems 106 with which a user performs exercises as part of physical fitness. In the illustrated example, the gym 104B includes a gym tracking server 116. Alternatively, the gym tracking server 116 may be hosted at or integrated into the performance system server 110. The gym tracking server 116 provides the portal 108 for the movement tracking systems 106, exchanges data with the performance system server 110, and acts to integrate the movement tracking systems 106 into the gym membership network 118 to facilitate linking a membership to the gym to the profile for the user maintained by the performance system server 110. As described below, a user registers with the operator of the gym to connect their profile managed by the performance system server 110 to their membership. This facilitates, for example, offering programs based on the membership. Through the portal 108, the user selects programs to workout targeted areas of the body and receive feedback for the programs completed. Because of the public setting, the information provided by the portal 108 at the gym 104B is more limited than the information provided by the portal at the clinic 104A and, in some examples, at the home 104C. The performance system server 110 collects this workout data to analyze. Notably, the user can elect to allow the portal 108 at the gym 104B to display all such information and the system allows for such flexibility in restricting data or information on a portal by portal basis.

In the illustrated example, the venues include a home 104C. While in the illustrated examples, the venue 104C is described as a home, it may be any other location in which a user lives, such as an assisted living facility or a dormitory. In the illustrated example, the home 104 includes one or more of the movement tracking systems 106 with which a user performs exercises as part of physical fitness and/or physical rehabilitation. In the illustrated example, the performance system server 110 provides the portal 108 for the movement tracking systems 106. Through the portal 108, the user selects programs to workout targeted areas of the body, receive feedback for the programs completed, review analytics calculated by the performance system server 110. In some examples, when the profile for the user maintained by the performance system server 110 is already connected to a gym membership, the user may be able to perform audio and/or visual communication over the network to, for example, a personal trainer associated with the gym. In such examples, the personal trainer may have a portal that has interaction with the portal 108 of the movement tracking systems 106 at the home 104C during the audio and/or visual communication to add, change, or delete various programs in accordance with the user's fitness goals. The information provided by the portal 108 at the home 104C may be configurable to adjust the amount of private information available. For example, the portal 108 displays physical rehabilitation programs and data, gym programs and data, and/or analytics computed by the performance system server 110.

The performance system server 110 maintains user profiles in the user database 120, such as maintaining information about memberships and/or associations. The performance system server 110 also communicates profile data to the portal 108; accordingly, to access those portals to provide profile data. The performance system server 110 also calculates analytics to quantify physical activity performed over the multiple venues 104A, 104B, 104C. The analytics may provide a trackable measure of performance over time to help, for example, identify needs for physical therapy and/or growth towards fitness/performance goals.

The movement tracking system 106 tracks movement of a user in a virtual space that corresponds to a physical space to analyze performance of a user compared to a selected program consisting of movements and exercises. Examples of movement tracking system 106 and the data collected for analysis by the performance system server 110 are described in U.S. Pat. No. 9,078,598 entitled, “Cognitive Function Evaluation and Rehabilitation Methods and Systems,” granted Jul. 14, 2015, U.S. Pat. No. 7,864,168, entitled Virtual Reality Movement System,” granted Jan. 4, 2011, and U.S. patent application Ser. No. 15/600,982, entitled “Method Of Evaluation Of Involving Physical Tasks And Neurological Input,” filed May 22, 2017, all of which are herein incorporated by reference.

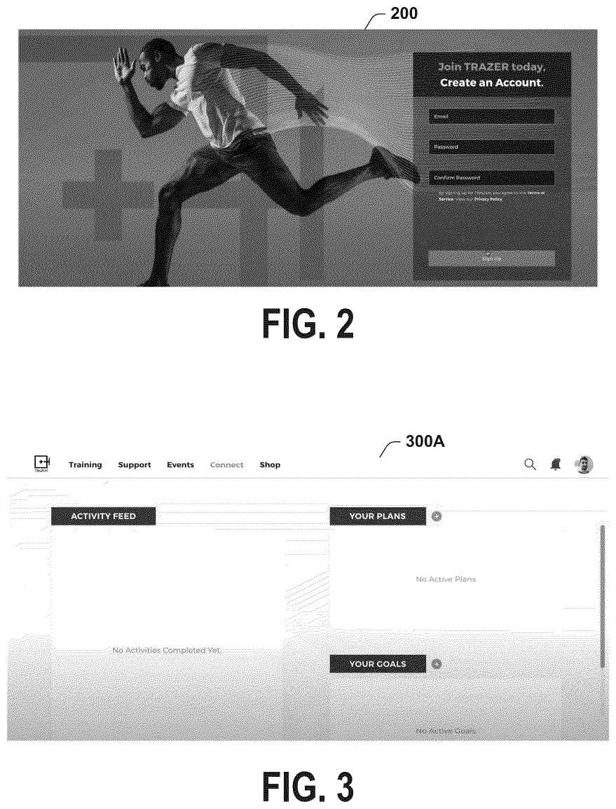

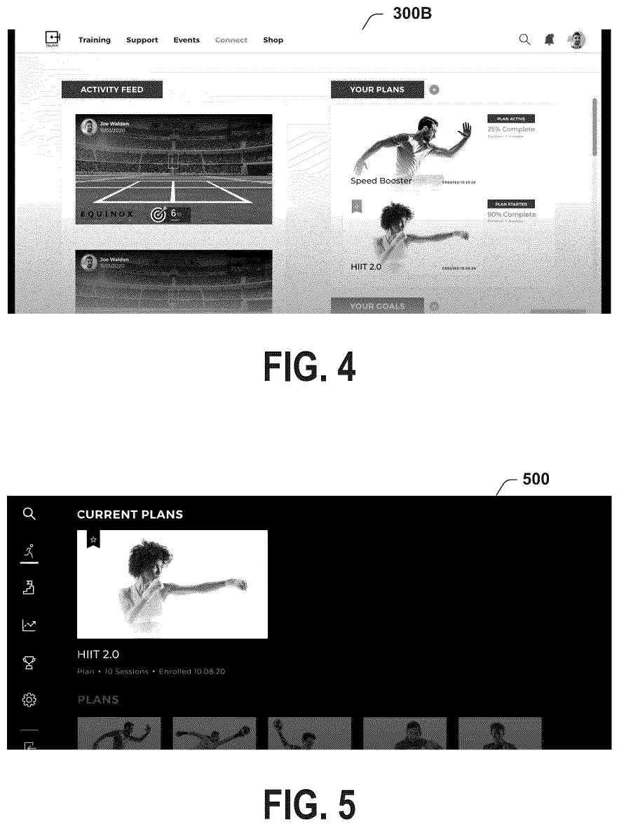

FIGS. 2-20 illustrate interfaces associated with one or more of portals 108 associated with the various venues 104A, 104B, and 104C. FIG. 2 illustrates an interface 200 for a login and/or account creation screen. During this process, the user may associate their profile with memberships to various facilities (e.g., gyms, employers, etc.) to facilitate access to programs related to those memberships. FIGS. 3, 4, and 5 illustrate an example interfacing 300A, 300B and 500 of a homepage displaying information regarding the profile of the user from the performance system server 110. These interfaces may be populated with content based on the user's profile and the portal through which the interface is being viewed. For example, FIG. 3 illustrates interface 300A as empty before being populated by information, if any, regarding the profile of the user from the performance system server 110. FIG. 4 illustrates interface 300B populated by information regarding the profile of the user from the performance system server 110. FIG. 5 illustrates interface 500 populated by information regarding the profile of the user from the performance system server 110.

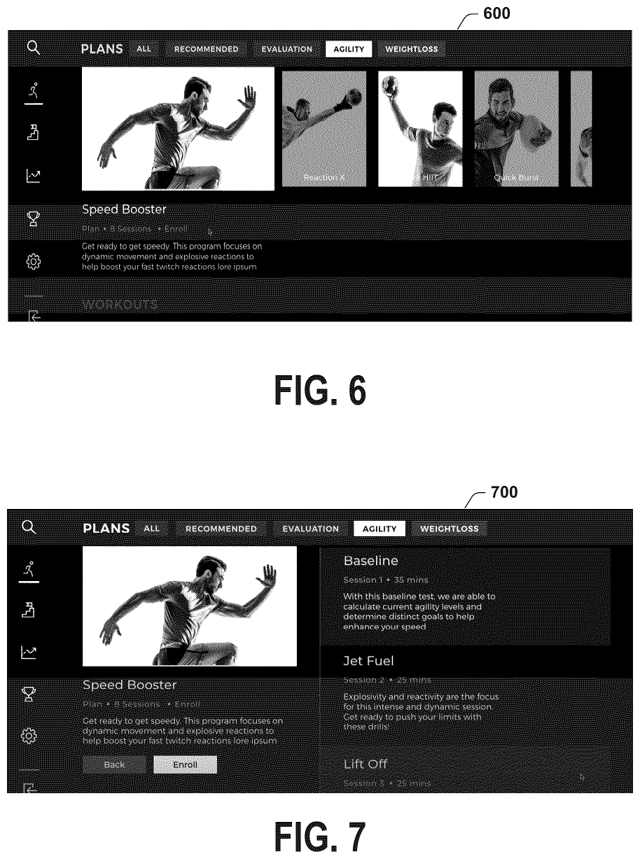

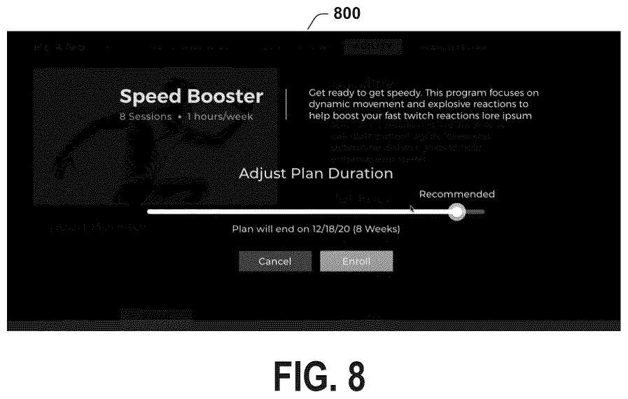

FIG. 6 illustrates an interface 600 to select a program to use with the movement tracking systems 106 to be added to the profile. FIG. 7 illustrates an interface 700 to facilitate viewing details of a program selected on interface 600. FIG. 8 illustrates an interface 800 to facilitate customizing a program selected on interface 600 to add the program to the profile of the user.

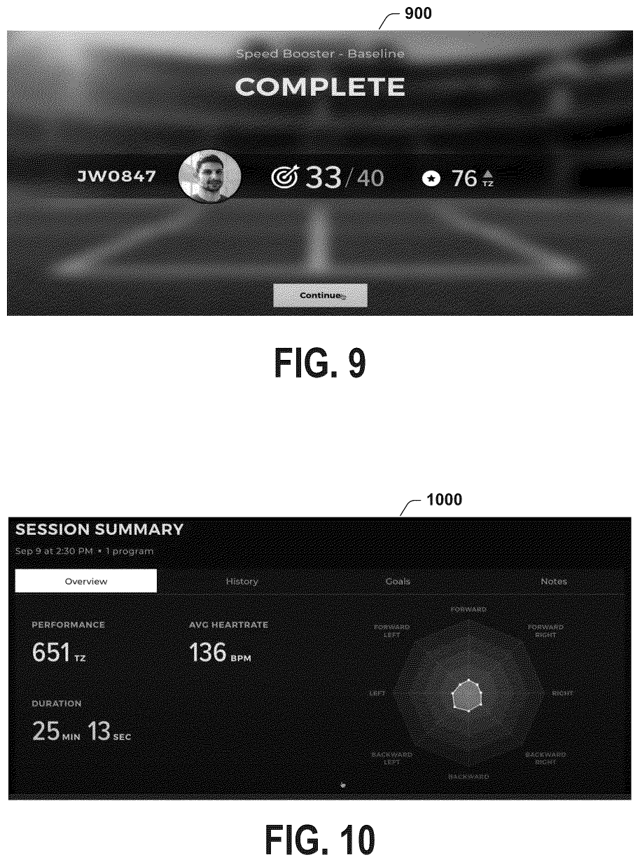

FIG. 9 illustrates an example interface 900 of a completion screen that shows details of various metrics, such as accuracy and/or performance rating (e.g., a quantitative analysis of the user's movements and/or exertion, etc.), etc. after a session of a program is complete. FIG. 10 illustrates an interface 1000 with a summary screen with another analysis of the session of the program with multiple statistics to be used to perform analytics on the performance of the user over time (e.g., the performance rating, heart rate, duration, a movement analysis graph, etc.).

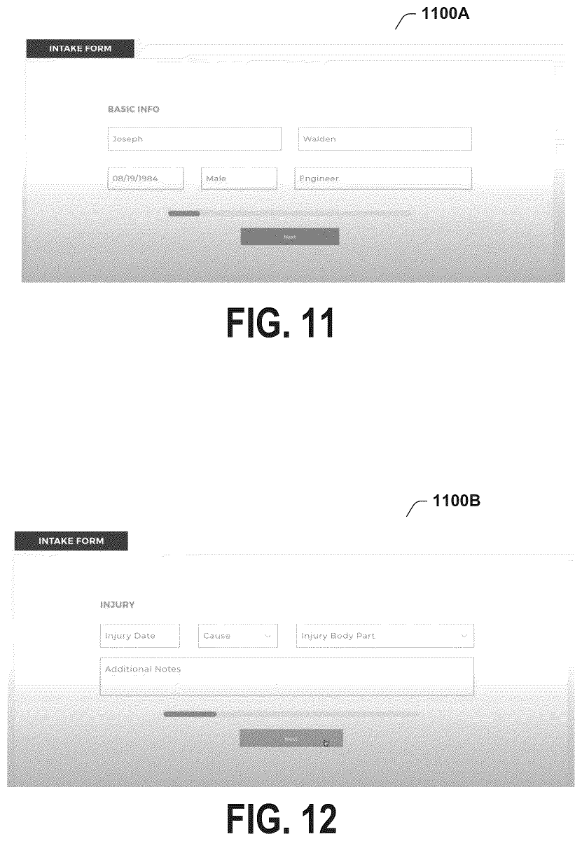

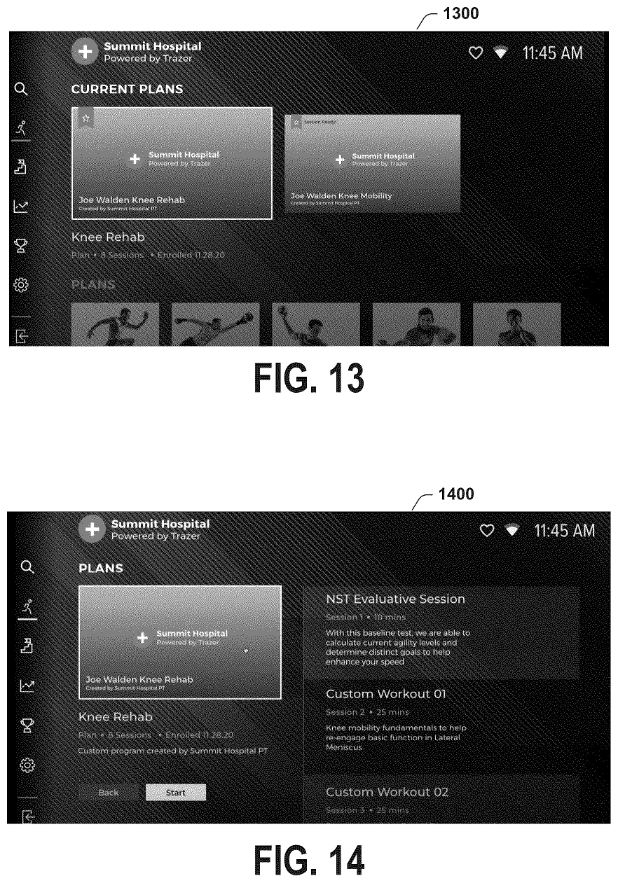

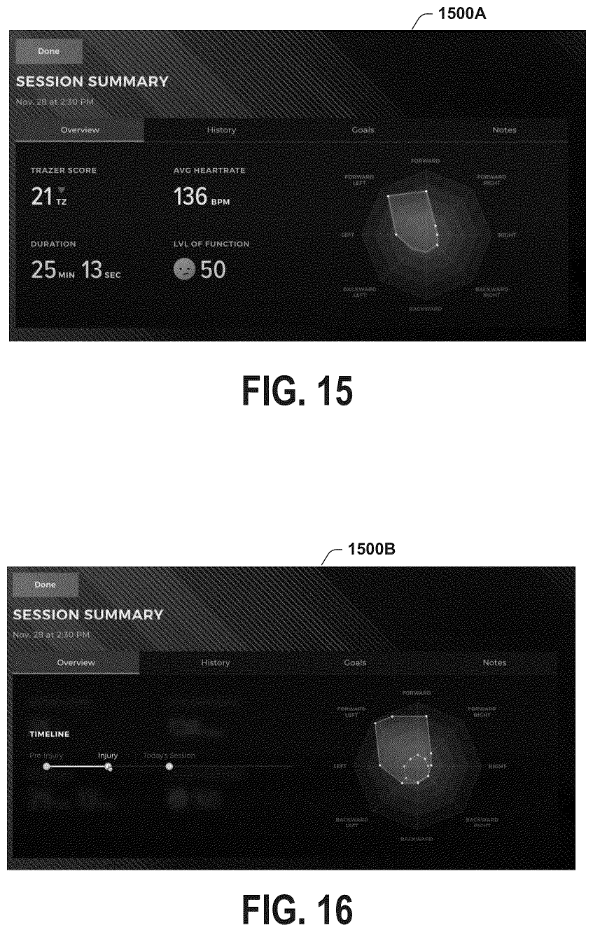

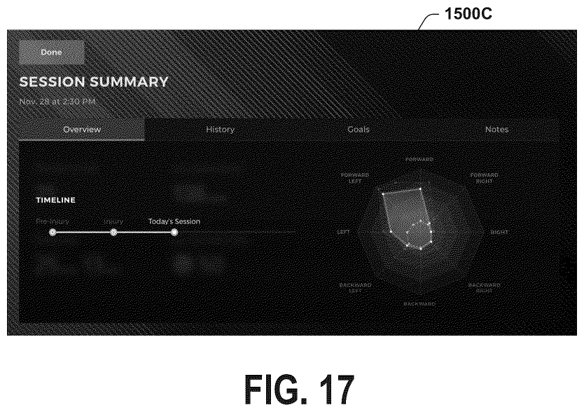

FIGS. 11 and 12 illustrate example interfaces 1100 and 1200 to enroll the user with a provider of the clinic 104A and associate the profile of the user with the provider of the clinic. A user may enroll, for example, after experiencing an injury. The provider may then, for example, associate the profile with the user data in the clinical network 114. FIG. 13 illustrates an example interface 1300 that provides information in the user's profile that is tagged or otherwise designated as being available to portals 108 associated with clinic 104A. FIG. 14 illustrates an interface 1400 with programs available to the user at the clinic 104A. FIG. 15 illustrates an interface 1500A that provides a summary of the session of the program with various metrics included (such as, the performance rating, the average heartrate, the duration, a quantitative measure of function of the anatomy being rehabilitated, the movement analysis graph, etc.). These analytics may be used to perform analytics on the performance of the user over time. FIG. 16 illustrates an interface 1500B that provides a comparison between two points in time (e.g., a time before an injury, and a time that coincides with the injury). FIG. 17 illustrates an interface 1500C that provides a comparison between multiple points in time (e.g., a time before an injury, a time that coincides with the injury, a time that coincides with the most current session, etc.).

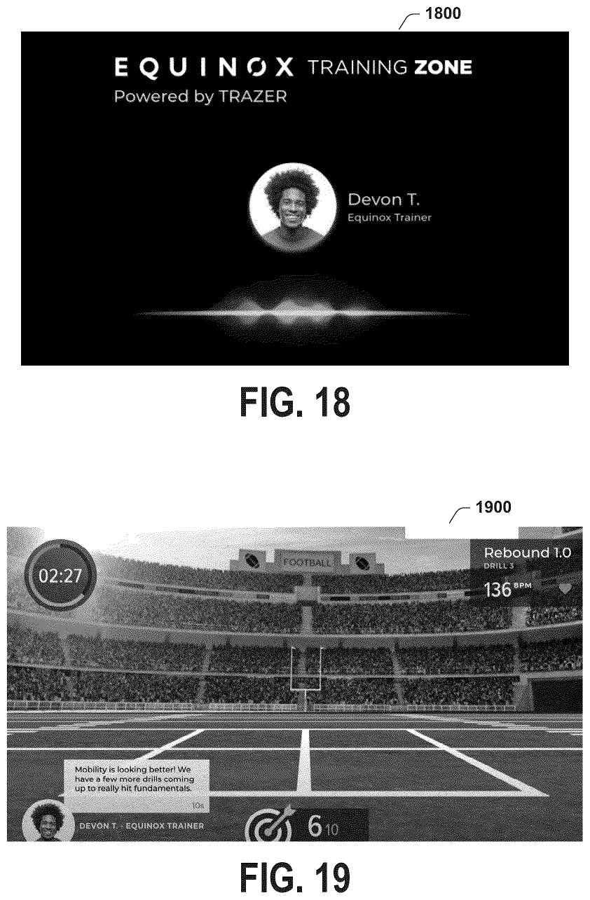

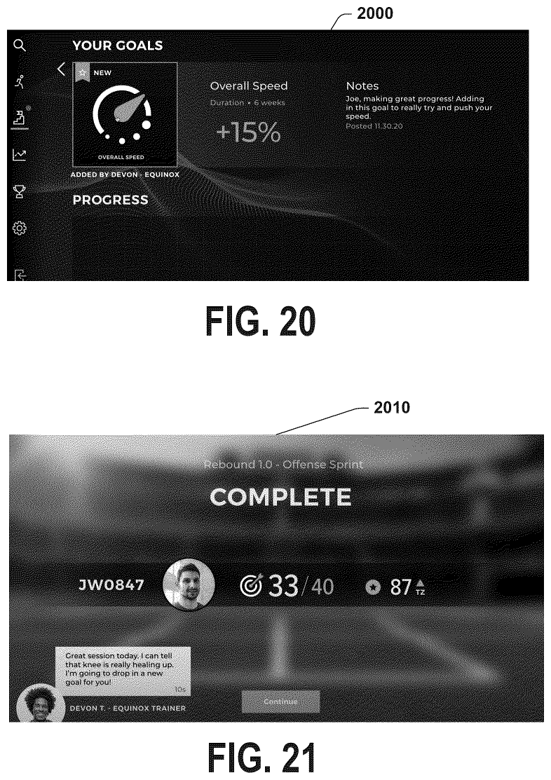

While at home 104A, the user may wish to access benefits of their gym membership. For example, the user may wish to access programs associated with their profile that are based on their membership to the gym 104B. FIGS. 18, 19, and 20 illustrate example interfaces 1800, 1900, and 2000 that provide audio and/or visual communication with a trainer associated with the gym 104B. FIG. 18 illustrates the interface 1800 that provides audio and/or visual communication with the trainer that may be accessed through the portal 108 at the home 104C. FIG. 19 illustrates the interface 1900 that provides a session of a selected program with indicia of the audio and/or visual communication with the trainer that may be accessed through the portal 108 at the home 104C. FIG. 20 illustrates the interface 2000 that provides analytical feedback and/or feedback from the trainer regarding the just completed session. FIG. 21 illustrates the interface 2010 that provides analytical feedback and/or feedback regarding the just completed session or activity.

Although the embodiments of the present invention have been illustrated in the accompanying drawings and described in the foregoing detailed description, it is to be understood that the present disclosure is not to be limited to just the embodiments disclosed, but that the disclosure described herein is capable of numerous rearrangements, modifications and substitutions without departing from the scope of the claims hereafter. The claims as follows are intended to include all modifications and alterations insofar as they come within the scope of the claims or the equivalent thereof.

1. A system, comprising:
a profile database that stores profile data associated with a user; and
a performance system server communicatively coupled to one or more movement tracking systems to collect and analyze movement of the user at one or more venues to provide physical assessment of the user, stored in the profile database along with collected movement information, wherein
the one or more movement tracking systems tracks the movement of the user in a virtual space that corresponds to a physical space and includes a portal for accessing the profile data.

2. The system of claim 1, wherein the performance system server provides a unified platform for analytics to quantitatively evaluate performance of the user across different activities at the one or more venues.

3. The system of claim 1, wherein the profile data associated with the user comprises biographic data, sports and fitness medical history, treatment history, exercise history, analytical data, membership information, or association information.

4. The system of claim 1, further comprising a clinical tracking server at the one or more venues if the one or more venues is a rehabilitation clinic, or alternatively the clinical tracking server is integrated into the performance system server, wherein
the clinical tracking server provides, through the performance system server, the portal for the one or more movement tracking systems, exchanges data with the performance system server, and acts to integrate the one or more movement tracking systems into a clinical network that contains clinic specific data used by the portal such as billing information, medical history, insurance, or other clinic specific data.

5. The system of claim 1, further comprising a gym tracking server at the one or more venues if the one or more venues is a gym or a fitness center, or alternatively the gym tracking server is integrated into the performance system server, wherein
the gym tracking server provides, through the performance system server, the portal for the one or more movement tracking systems, exchanges data with the performance system server, and acts to integrate the one or more movement tracking systems into a gym membership network to facilitate linking a membership to the gym or the fitness center to a profile for the user maintained by the performance system server.

6. The system of claim 1, wherein if the one or more venues is a home or a location of the user, the home or the location of the user includes the one or more movement tracking systems with the portal provided by the performance system server, and wherein the portal from the home or the location of the user provides an interface to access content available to any of the portal connected the one or more movement tracking systems, associated with the user, at the one or more venues such as gym-specific content or clinically specific content.

7. The system of claim 1, wherein the portal is configured to provide a means to adjust amount of private information available and communicate with other portals such as portals of a trainer or a clinician.

8. The system of claim 1, wherein the portal provides an interface for account creation, logging in, selectively displaying the profile data, selecting one or more fitness programs, displaying details for the selected fitness programs, customizing the selected fitness programs, displaying a completion screen showing details of various metrics from completed fitness programs, displaying a summary screen with analysis and statistics, enrolling the user with a provider of a clinic, display data associated with the clinic, and providing audio visual communication with a trainer or a clinician.

9. The system of claim 1, wherein the system is a cloud-based system and the physical assessment from the one or more venues is stored in the profile database in the cloud-based system.

10. A method, comprising:
storing, in a profile database of a system, profile data associated with a user;
communicatively coupling, by a performance system of the system, with one or more movement tacking systems that tracks movement of the user in a virtual space that corresponds to a physical space and includes a portal for accessing the profile data;
collecting and analyzing, by a performance system server of the system, the movement of the user at one or more venues to provide physical assessment of the user; and
storing, in the profile database of the system, collected movement information and the physical assessment of the user.

11. The method of claim 10, further comprising providing, by the performance system of the system, a unified platform for analytics to quantitatively evaluate performance of the user across different activities at the one or more venues.

12. The method of claim 10, wherein the storing of the profile data associated with the user comprises storing biographic data, sports and fitness medical history, treatment history, exercise history, analytical data, membership information, or association information.

13. The method of claim 10, further comprising, if the one or more venue is a clinic,
providing, by clinical tracking server through the performance system server, the portal for the one or more movement tracking systems;
exchanging, by the clinical tracking server, data with the performance system server; and
acting, by the clinical tracking server, to integrate the one or more movement tracking systems into a clinical network that contains clinic specific data used by the portal such as billing information, medical history, insurance, or other clinic specific data.

14. The method of claim 10, further comprising, if the one or more venue is a gym or fitness center,
providing, by a gym or fitness tracking server through the performance system server, the portal for the one or more movement tracking systems;
exchanging, by the gym or fitness tracking server, data with the performance system server; and
acting, by the gym or fitness tracking server, to integrate the one or more movement tracking systems into a gym membership network to facilitate linking a membership to the gym or the fitness center to a profile for the user maintained by the performance system server.

15. The method of claim 10, further comprising, if the one or more venues is a home or a location of the user, providing, by the performance system server, the portal for the one or more movement tracking systems located in the home or the location of the user, wherein the portal from the home or the location of the user provides an interface to access content available to any of the portal connected to the one or more movement tracking system, associated with the user, at the one or more venues such as gym-specific content or clinically specific content.

16. The method of claim 10, further comprising providing, by the system, a means through the portal for adjusting amount of private information available and communicating with other portals such as portals of a trainer or a clinician.

17. The method of claim 10, further comprising, providing, by the performance system server, the portal for providing an interface for account creation, logging in, selectively displaying the profile data, selecting one or more fitness programs, displaying details for the selected fitness programs, customizing the selected fitness programs, displaying a completion screen showing details of various metrics from completed fitness programs, displaying a summary screen with analysis and statistics, enrolling the user with a provider of a clinic, display data associated with the clinic, and providing audio visual communication with a trainer or a clinician.

18. The system of claim 10, wherein the system is a cloud-based system, and the storing of the profile data comprises storing the profile data in the profile database in the cloud-based system.

19. A movement tracking apparatus, comprising:
a camera system for tracking movement of a user responsive to a motion prompt; and
a portal to display the motion prompt and profile data associated with the user.

20. The movement tracking apparatus of claim 19, wherein the portal display different parts of the profile data depending on whether the movement tracking apparatus is part of a clinic and the portal is a clinical portal, a gym or fitness center and the portal is a gym or fitness portal, or a home and the portal is a home portal.
